(19) United States
(12) Patent Application Publication
(10) Pub. No.: US 2025/0073450 A1
(43) Pub. Date: Mar. 6, 2025

(54) Common Mode Interference Suppression In An Amplifier Circuit For A Neuromodulation Device

(71) Applicant: IMEC VZW, Leuven (BE)

(72) Inventors: Chutham Sawigun, Leuven (BE); Carolina Mora Lopez, Heverlee (BE); Xiaolin Yang, Leuven (BE); Joan Aymerich, Leuven (BE)

(21) Appl. No.: 18/818,054

(22) Filed: Aug. 28, 2024

(30) Foreign Application Priority Data
Aug. 29, 2023 (EP) 23193810.1

(51) Int. Cl. A61N 1/02; A61N 1/36; H03F 3/45

(52) U.S. Cl. CPC A61N 1/025; A61N 1/36017; A61N 1/36025; A61N 1/36031; H03F 3/45475; H03F 2200/129; H03F 2203/45512; H03F 2203/45524

(57) ABSTRACT

The present disclosure provides a neuromodulation device that comprises at least one amplifier circuit that suppresses a common mode (CM) voltage signal in the input voltage signal. The amplifier circuit comprises an input stage to receive the input voltage signal, and a differential transconductor to provide an output current signal based on a DM voltage signal in the input voltage signal. The transconductor is provides a first CM voltage signal tapped after a non-inverting input, and a second CM voltage signal tapped after am inverting input, to CM amplifier of the amplifier circuit. The CM amplifier combines the first CM voltage signal with the second CM voltage signal, amplifies the combined CM voltage signal with an inverting gain, and provides the inverted CM voltage signal back to the non-inverting input and the inverting input of the transconductor for enabling the CM suppression.

### CROSS-REFERENCE TO RELATED APPLICATIONS

The present application is a non-provisional patent application claiming priority to application No. EP 23193810.1, filed Aug. 29, 2023, the contents of which are hereby incorporated by reference.

### FIELD OF THE DISCLOSURE

The present disclosure provides a neuromodulation device for measuring a biological electrical signal in response to a neural modulation of biological tissue. The amplifier circuit provided by this disclosure is configured to suppress a common mode (CM) voltage signal in the input voltage signal, and consequently to reduce CM interference (CMI).

### BACKGROUND

A bi-directional (i.e., closed loop) neural interface may be used by a device to perform both neural stimulation and recording. Such a device enables neuromodulation—i.e., is referred to as a neuromodulation device—which is useful for neuroscience studies and treatments of neurological disorders.

For performing the neural recording, an amplifier circuit (also referred to as neural amplifier) is required, and should be able to perform low-noise small-signal amplification, while at the same time rejecting a large electrode DC offset (EDO). To achieve the desired EDO rejection ability, capacitively coupled architectures have been widely adopted for such amplifier circuits, since the input capacitors exhibit infinite impedance at DC, and thus prevent the EDO from being amplified. To achieve the low-noise performance, a differential inverter-based transconductor is advantageously used in the amplifier circuit, since it provides a higher transconductance than other well-known types of transconductor (e.g., simple wide-swing, or folded cascode).

An example of a conventional capacitively coupled amplifier circuit that may be used in a neuromodulation device is shown in FIG. 1(a). The amplifier circuit has an inverter as input stage, which is shown in FIG. 1(b). When recording in a controlled environment, the amplifier circuit of FIG. 1(a) performs reasonably well, since there is no severe CMI disturbing its performance. However, for a bi-directional neural interface, the amplifier circuit may suffer from CMI due to its limited CM range (CMR).

In particular, during performing the neural stimulation, depending on the distance between the place of stimulation to recording electrodes, and on how strong the stimulation signal is, the stimulation signal can partially propagate to the input (recording and reference electrodes) of the amplifier circuit of FIG. 1(a), as a stimulation waveform-like CM voltage (V_{CM}, also referred to as CM voltage signal). A high-pass cut-off frequency fₕ in the amplifier circuit is defined by the resistor R_{f} and the capacitor C_{f}, namely as fₕ=1/(2πC_{f}R_{f}), and is typically set below 1 Hz. The VCM can travel to appear at the input of the amplifier circuit, if the stimulation frequency is higher than fₕ (and it mostly is). Considering the amplifier's input stage shown in FIG. 1(b), and due to its stacking nature, the CMR of the amplifier is limited to V_{DD}−V_{SS}−(V_{B1}+V_{B2}+VGSₙ+VSGₚ), where V_{B1} and V_{B2} are minimum voltage drops required by tail current sources I_{B1} and I_{B2}, respectively. The other parameters have their own usual meanings. The V_{CM} can be in the range of 50-300 mV or even greater. For these reasons, the input stage of FIG. 1(b) cannot always handle the VCM, and consequently the amplification of the amplifier circuit of FIG. 1(a) may fail.

To address this problem, a CM feedforward (CMFF) amplifier may be inserted into the amplifier circuit, as shown in FIG. 1(c). The CMFF amplifier contains a transconductor (A), a pair of CM detector capacitors C_{ic}, a feedback capacitor C_{cf}, a resistor R_{cf}, and current stealing capacitors C_{oc}. This amplifier circuit detects and amplifies the V_{CM} with an AC gain of −2C_{ic}/C_{cf}. The input CM current flowing through Cᵢₙ (i.e., I_{CM}) can be calculated by setting the CM input terminal of the amplifier circuit to ground (i.e., Vᵢ+=Vᵢ−=0). The CMFF can then be designed to steal I_{CM} away from that node through capacitors C_{oc} under the condition that C_{oc}=CᵢₙC_{cj}/(2C_{ic}). By doing so, the input CM voltage fluctuations at the input terminal of the amplifier circuit can be minimized. The inverter based input stage can remain functioning for V_{CMpp}>CMR.

However, the additional CMFF amplifier shown in FIG. 1(c) adds more input capacitor C_{ic} in addition to the original Cᵢₙ, thereby degrading the input impedance of the amplifier circuit. In case C_{ic}=Cᵢₙ is set, the input impedance may be halved. This resulting smaller input impedance is not desirable, since it attenuates the signal and enlarges the input-referred noise (IRN). To compensate for this impedance degradation, the amplifier circuit of FIG. 1(c) may further incorporate an impedance boosting network, which leads to additional hardware complexity. However, especially for multichannel closed-loop neural interfaces, the chip area per channel needs to be kept tiny, so as to accommodate several hundreds of channels or more within a reasonable total chip area. The large chip area per channel as required by the design of FIG. 1(c) may thus not be acceptable.

### SUMMARY

The present disclosure provides an amplifier circuit for a neuromodulation device, in particular, to overcome the above-mentioned issues. In some example embodiments, the present disclosure provides for the suppression of a CM voltage signal and reduction of CMI, while keeping the size of the amplifier circuit small. A further objective is to avoid input impedance degradation. In some example embodiments, the present disclosure provides for the performance of the amplifier circuit for low-noise small-signal amplification, and that the amplifier circuit at the same time rejects large EDO. Another objective of this disclosure is to provide an improved neuromodulation device.

In some example embodiments, the present disclosure provides an amplifier circuit for amplifying a biological electrical signal, the amplifier circuit comprising: an input stage with two input terminals, configured to receive an input voltage signal, which is based on the biological electrical signal and comprises a CM voltage signal and a differential mode (DM) voltage signal; a differential transconductor comprising a non-inverting input and an inverting input, each connected to one of the two input terminals, and comprising an output for providing an output current signal based on the DM voltage signal; wherein the transconductor is configured to provide a first CM voltage signal, which is tapped after the non-inverting input, and a second CM voltage signal, which is tapped after the inverting input; and a CM amplifier configured to combine the first CM voltage signal with the second CM voltage signal to obtain a combined CM voltage signal, amplify the combined CM voltage signal with an inverting gain to obtain an inverted CM voltage signal, and provide the inverted CM voltage signal to the non-inverting input and the inverting input of the transconductor.

In some example embodiments, the CM amplifier of the amplifier circuit suppresses the CM voltage signal in the input signal and reduces CMI. Since the amplifier circuit comprises the CM amplifier after the transconductor, input impedance degradation is avoided. Also, the size of the amplifier circuit can be smaller than, for example, the amplifier circuit shown in FIG. 1(c). Due to the differential transconductor, a good low-noise performance of the amplifier circuit can be achieved.

In some example embodiments, the amplifier circuit comprises: a first capacitive feedback line configured to feedback the inverted CM voltage signal from the CM amplifier to the non-inverting input of the transconductor; and a second capacitive feedback line configured to feedback the inverted CM voltage signal from the CM amplifier to the inverting input of the transconductor; wherein each of the first and the second capacitive feedback line comprises a first capacitor.

In some example embodiments of the amplifier circuit, each of the non-inverting input and the inverting input of the transconductor is connected to one of the two input terminals of the input stage via a respective second capacitor; and the second capacitor has a higher capacitance than the first capacitor.

In some example embodiments, the capacitive feedback from the CM amplifier as disclosed leads to low noise in the amplifier circuit.

In some example embodiments of the amplifier circuit, the transconductor comprises: a first transistor connected with its gate to the non-inverting input and coupled with its source to a supply voltage, V_{DD}; a second transistor connected with its gate to the inverting input, connected with its source to the source of the first transistor, and coupled with its source to V_{DD}; a third transistor connected with its gate to the non-inverting input, connected with its drain to the drain of the first transistor, and coupled with its source to a ground voltage; and a fourth transistor connected with its gate to the inverting input, connected with its drain to the drain of the second transistor, connected with its source to the source of the third transistor, and coupled with its source to V_{SS}; wherein the first CM voltage signal is tapped between the sources of the first and the second transistor, and the second CM voltage signal is tapped between the sources of the third and the fourth transistor.

In some example embodiments, the amplifier circuit is configured such that a tail current of the source-connected third and fourth transistor is higher than a tail current of the source-connected first and second transistor in use of the amplifier circuit. This allows folded-cascode branches to deliver the output current signal of the amplifier circuit.

In some example embodiments of the amplifier circuit, the CM amplifier comprises a summing junction at which the first CM voltage signal and the second CM voltage signal are combined into the combined CM voltage signal; wherein each of the first CM voltage signal and the second CM voltage signal is connected to the combination point via a respective third capacitor.

In some example embodiments of the amplifier circuit, the CM amplifier further comprises a gain circuit including a second transconductor, a fourth capacitor and a first resistor; and the gain circuit is configured to amplify the combined CM voltage signal with the inverting gain to produce the inverted CM voltage signal.

In some example embodiments, the amplifier circuit further comprises a DM feedback circuit configured to provide a feedback voltage signal, which is based on the output current signal of the transconductor, to the non-inverting input and the inverting input of the transconductor.

In some example embodiments of the amplifier circuit, the DM feedback circuit comprises a first capacitive feedback path and a second capacitive feedback path; and each capacitive feedback path comprises a respective fifth capacitor that is connected in parallel to a respective second resistor. The example capacitively coupled feedback architecture achieves the desired EDO rejecting abilities of the amplifier circuit.

In some example embodiments, the amplifier circuit further comprises a conversion circuit configured to convert the output current signal of the transconductor into the feedback voltage signal.

In some example embodiments of the amplifier circuit, the biological electrical signal is an electroencephalogram, (EEG) signal or a stimulation-based neural signal.

In some example embodiments of this disclosure provides a neuromodulation device for measuring one or more biological electrical signals in response to a neural modulation of biological tissue, wherein the neuromodulation device comprises one or more amplifier circuits of the first aspect or any implementation thereof.

In some example embodiments, the neuromodulation device further comprises an array of electrodes comprising a first set of electrodes and a second set of electrodes, wherein each electrode of the first set is connected to a respective amplifier circuit of the one or more amplifier circuits, and wherein each electrode of the second set is configured to cause a respective neural modulation of the biological tissue. That is, some electrodes can be used for recording, and some other electrodes for stimulating, which may be beneficial for the performance of the neuromodulation device.

In some example embodiments of the neuromodulation device, each electrode of the second set comprises a stimulator, which is configured to provide a stimulation signal to cause the neural modulation of the biological tissue.

In some example embodiments, the neuromodulation device comprises multiple of the amplifier circuits; wherein the amplifier circuits share a single CM amplifier; and the CM amplifier is configured to provide, for each of the amplifier circuits, a respective inverted CM voltage signal to the non-inverting input and the inverting input of the transconductor of that amplifier circuit. This allows reducing the size of the neuromodulation device significantly, in particularly, reducing the area per channel.

In some example embodiments, the neuromodulation device further comprises one or more shared buffers connected to the multiple amplifier circuits and to the single CM amplifier, wherein the output of each amplifier circuit is connected by a respective switch to the shared buffers; wherein the neuromodulation device is configured to sequentially operate the switches, which causes the multiple amplifier circuits to sequentially output their respective current signal and causes the CM amplifier to sequentially provide the respective inverted CM voltage signal to the respective transconductor of one of the amplifier circuits. The shared buffers allow multiplexing the multiple channels.

In some example embodiments, the neuromodulation device further comprises a bi-directional neural interface comprising at least one needle; wherein the neural interface is configured to provide the neural stimulation to the biological tissue, to receive the one or more biological electrical signals, and to provide them to the one or more amplifier circuits.

### BRIEF DESCRIPTION OF THE DRAWINGS

The above, as well as additional objects, features and advantages of the present disclosure, will be better understood through the following illustrative and non-limiting detailed description, with reference to the appended drawings. In the drawings like reference numerals will be used for like elements unless stated otherwise.

FIG. 1 shows exemplary amplifier circuits for a neuromodulation device, in particular, (a) without CM suppression and (b) with CM suppression circuitry.

FIG. 2 shows a general architecture of an amplifier circuit for a neuromodulation device according to this disclosure.

FIG. 3 shows an exemplary amplifier circuit for a neuromodulation device according to this disclosure.

FIG. 4 shows the transistor-level of the amplifier circuit of FIG. 3.

FIG. 5 compares output voltage signals of an exemplary amplifier circuits having (a) no CM suppression and (b) a CM amplifier for CM suppression according to this disclosure.

FIG. 6 shows an exemplary neuromodulation device according to this disclosure.

FIG. 7 shows an example of a neuromodulation device according to this disclosure, which comprises an array of electrodes and a needle as bi-directional neural interface.

FIG. 8 shows a multichannel recording arrangement according to this disclosure with a shared CM amplifier and shared buffers.

FIG. 9 shows a simulated channel discrete Fourier transform (DFT) of a 16-channel architecture of the multichannel recording arrangement according to this disclosure, in particular, (a) without reset switches and (b) with reset switches.

### DETAILED DESCRIPTION

In cooperation with attached drawings, the technical contents and detailed description of the embodiments are described thereinafter according to an example embodiment, being not used to limit the claimed scope. This disclosure may be embodied in many different forms and should not be construed as limited to the embodiments set forth herein; rather, these embodiments are provided for thoroughness and completeness, and fully convey the scope of the concepts for which protection is sought.

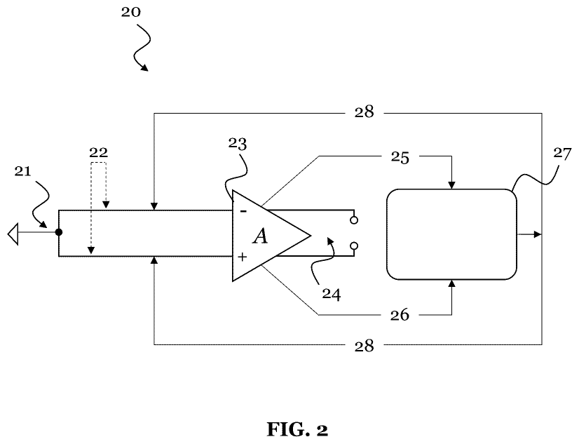

FIG. 2 shows an amplifier circuit 20 according to this disclosure. The amplifier circuit 20 is configured to amplify a biological electrical signal, for instance, as received in response to a neural modulation of biological tissue. That is, the biological electrical signal may be a stimulation-based neural signal. The biological electrical signal may, for example, be an EEG signal. The amplifier circuit 20 is suitable for use in a neuromodulation device or a recording arrangement. Due to its low-noise performance and efficient CM suppression, the amplifier circuit 20 is especially suited for a neuromodulation device using a bi-directional neural interface for neural stimulation and signal recording.

The amplifier circuit 20 shown in FIG. 2 comprises an input stage 21 with two input terminals, a differential transconductor 23, and a CM amplifier 27.

The input stage 21 is configured to receive an input voltage signal 22, which is based on the biological electrical signal that the amplifier circuit 20 is intended to amplify. For instance, the input voltage signal 22 may be derived from the biological electrical signal, or may be the biological electrical signal, or the biological electrical signal may be signal processed to obtain the input voltage signal 22. The input voltage signal 22 comprises a CM voltage signal and a DM voltage signal. The DM voltage signal is the signal component, which the amplifier circuit 20 is supposed to amplify, while the CM voltage signal is the signal component that the amplifier circuit 20 is designed to suppress.

The differential transconductor 23 comprises a non-inverting input and an inverting input, like a differential amplifier. Each input of the transconductor 23 is connected to one of the two input terminals, i.e., the input voltage signal 22 is provided to the differential transconductor 23. The transconductor 23 further comprises an output 24, which is configured to provide an output current signal of the amplifier circuit 20, wherein the output current signal is based on the DM voltage signal. For instance, the output current signal may amplify the DM voltage signal. The amplifier circuit 20 is further configured to suppress the CM voltage signal in the input voltage signal 22 from appearing or at least substantially influencing the output current signal.

To this end, the transconductor 23 is configured to provide a first CM voltage signal 25 and a second CM voltage signal 26 to the CM amplifier 27. The first CM voltage signal is tapped after the non-inverting input, for instance, between the non-inverting input and the output 24 of the transconductor 23. The second CM voltage signal 26 is tapped after the inverting input, for instance, between the inverting input and the output 24 of the transconductor 23. Exemplary details where the CM voltage signals 25, 26 are tapped are provided below.

The CM amplifier 27 is configured to combine the first CM voltage signal 25 with the second CM voltage signal 26, so as to obtain a combined CM voltage signal. For instance, the first and second CM voltage signal 25, 26 may be summed. Then, the CM amplifier 27 is configured to amplify the combined CM voltage signal with an inverting gain, so as to obtain an inverted CM voltage signal 28. This inverted CM voltage signal 28 is then provided back to the non-inverting input and the inverting input of the transconductor 23, respectively, as indicated in FIG. 2. This feedback leads to a suppression of the CM voltage signal in the output current signal of the transconductor 23.

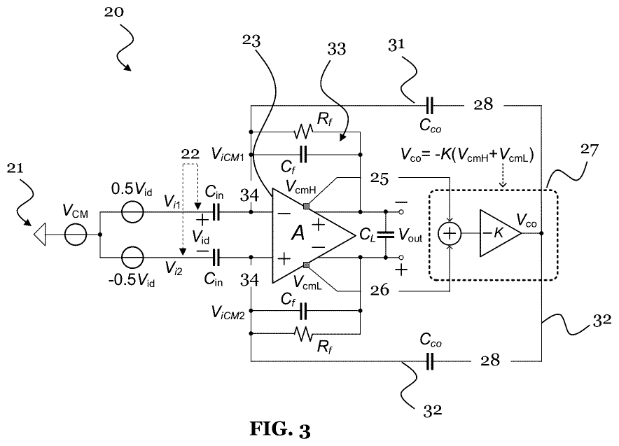

FIG. 3 shows an exemplary and more detailed amplifier circuit 20 according to this disclosure, which builds on the amplifier circuit 20 shown in FIG. 2. Same components in FIG. 2 and FIG. 3 share the same reference signs and function likewise.

The amplifier circuit 20 shown in FIG. 3 comprises capacitive feedback lines to provide the inverted CM voltage signal 28 back to the inputs of the transconductor 23. The feedback lines of the amplifier circuit include a first capacitive feedback line 31, which is configured to feedback the inverted CM voltage signal 28 from the CM amplifier 27 to the non-inverting input of the transconductor 23, and a second capacitive feedback line 32, which is configured to feedback the inverted CM voltage signal 28 from the CM amplifier 27 to the inverting input of the transconductor 23. Both capacitive feedback lines 31 and 32 comprise a capacitor C_{co}.

The amplifier circuit 20 comprises further capacitors. For instance, the amplifier circuit 20 comprises a capacitor C_{L} at the output 24, in particular, between two output terminals of the output 24. Further, the amplifier circuit 20 comprises two capacitors Cᵢₙ. Namely, both the non-inverting input and the inverting input of the transconductor 23 may be connected via one capacitor Cᵢₙ to the input stage 21. The capacitors Cᵢₙ may be selected such that they have (each) a higher capacitance than the capacitors C_{co}.

The amplifier circuit 20 further comprises a DM feedback circuit 33, which is configured to provide a feedback voltage signal 34, which is based on the output current signal of the transconductor 23—which is again based on the DM voltage signal in the input voltage signal 22—to the two inputs of the transconductor 23. The DM feedback circuit 33 comprises a first and a second capacitive feedback path, wherein each feedback path comprises a capacitor C_{f} and a resistor R_{f} that is connected in parallel to C_{f}.

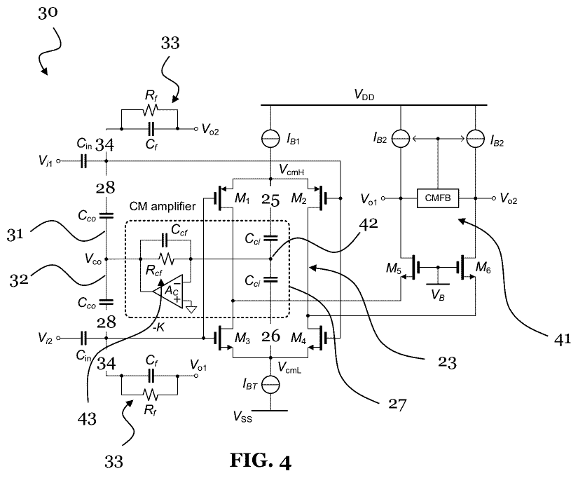

FIG. 4 shows an exemplary transistor-level implementation of an amplifier circuit 20 according to the present disclosure, for example, the amplifier circuit 20 shown in FIG. 3. Same components in FIG. 3 and FIG. 4 share the same reference signs and function likewise.

As can be seen in FIG. 4, the transconductor 23 comprises four transistors M₁, M₂, M₃, and M₄. The first transistor M₁ is connected with its source to the source of the second transistor M₂. Similarly, the third transistor M₃ is connected with its source to the source of the fourth transistor M₄. Accordingly, the pair M₁-M₂ and the pair M₃-M₄ is, respectively, a source-connected transistor pair. These two transistor pairs are connected to each other. In particular, the first transistor M₁ is connected with its drain to the drain of the third transistor M₃, and similarly the second transistor M₂ is connected with its drain to the drain of the fourth transistor M₄.

Further, the gates of the first transistor M₁ and the third transistor M₃ are both connected to the non-inverting input, and the gates of the second transistor M₂ and the fourth transistor M₄ are both connected to the inverting input. Moreover, the connected sources of the first transistor M₁ and the second transistor M₂ are further coupled to V_{DD}, e.g., via a current source, and the connected sources of the third transistor M₃ and the fourth transistor M₄ are further coupled to V_{SS}, e.g., via a current source. Notably, in this disclosure “connected” typically denotes a direct electrical connection between two components, while “coupled” denotes and indirect connection, for instance, via one or more other components.

In the exemplary amplifier circuit 20 of FIG. 4, the first CM voltage signal 25 is tapped between the sources of the first and the second transistor M₁, M₂, and the second CM voltage signal 26 is tapped between the sources of the third and the fourth transistor M₃, M₄, for instance, at the respective coupling connection of these sources to either V_{DD} and V_{SS}.

These CM voltage signals 25, 26 are provided to the CM amplifier 27, in particular, are routed to a summing junction 42. The lines carrying the first CM voltage signal 25 and the second CM voltage signal 26 may be respectively connected to the summing junction 42 via a respective capacitor C_{ci}. At the summing junction 42, the first CM voltage signal 25 and the second CM voltage signal 26 are combined, i.e., added together. This results in the combined CM voltage signal. The combined CM voltage signal is further provided to a gain circuit 43 of the CM amplifier 27. The gain circuit 43 includes a transconductor A_{C}, a capacitor C_{cf} and a resistor R_{cf} as shown, and is configured to amplify the combined CM voltage signal with the inverting gain (negative gain), which results in the inverted CM voltage signal 28. The inverted voltage signal 28 is provided via the capacitive feedback lines 31, 32 to the inverting and non-inverting input of the transconductor 23.

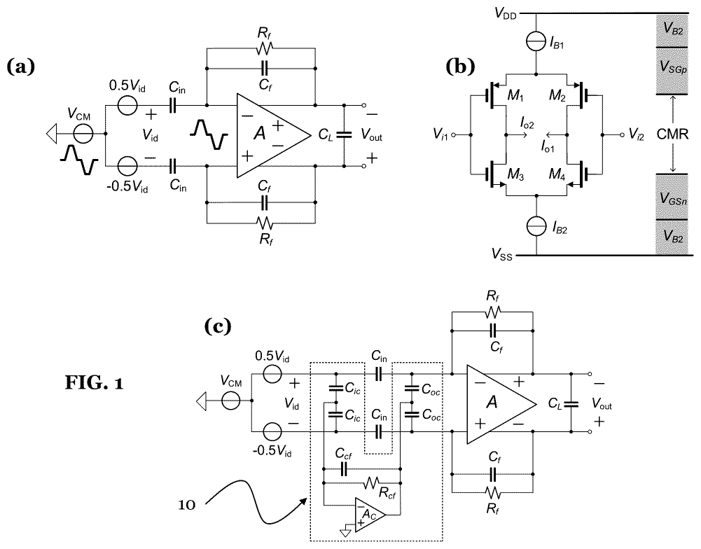

An underlying concept of the amplifier circuits 20 shown in the FIGS. 2-4 is that instead of detecting the CM voltage signal at the input stage 21 and performing feedforward cancellation—as shown for the exemplary amplifier circuit of FIG. 1(c)—the CM voltage signal of the input voltage signal 22 is allowed to enter the transconductor 23, and a feedback-based CM suppression is performed. This helps to avoid the undesired input impedance degradation.

The transconductor 23 (also labelled with A in the FIGS. 3 and 4 provides the two CM voltage signals V_{cmH} and V_{cmL}. These voltage signals are then combined and amplified with the inverting gain −K. The resulting output of the CM amplifier 27 is the inverted CM voltage signal 28 (also denoted V_{CO}), which is determined by:

V_{CO}=−KV_{ci}=−K(V_{cmH}+V_{cmL})=−KV_{iCM}.

In the above formula, V_{iCM1}=V_{iCM2}≅2V_{cmH}≅2V_{cmL}. The inverted CM voltage signal 28 is fed back to the input terminals of the transconductor 23, via the feedback lines 31, 32 including the capacitors C_{CO}. This feedback mechanism creates a large equivalent capacitance C_{EQ} to ground, as seen by the CM voltage signals which is defined by:

$${C}_{EQ} = 2 ( 1 + K ) {C}_{CO} .$$

With this large equivalent capacitance C_{EQ}, and for K>>1, the signal V_{iCM} can be calculated by:

$${V}_{iCM} = {V}_{CM} ( \frac{2 {C}_{in}}{2 {C}_{in} + {C}_{EQ}} ) ≅ {V}_{CM} ( \frac{{C}_{in}}{{C}_{in} + {KC}_{OC}} ) . \qquad ( 1 )$$

For the ideal case that K=∞, V_{iCM} becomes zero, meaning that there are no CM voltage fluctuations at the inputs of the transconductor 23. In practice, configuring C_{CO}<<Cᵢₙ may be beneficial for noise reasons. For example, a practical value of K>50 is desirable. For DM operation, V_{CO} may be seen as AC ground, and the pair of capacitors C_{CO} (without gain) may be virtually grounded.

In the transistor-level implementation shows in FIG. 4, the tail current of source-coupled transistor pair M₃-M₄ may be set higher than the tail current of the transistor pair M₁-M₂) as I_{BT}−I_{B1}+2I_{B2}. This is to allow folded-cascode branches (M₅−I_{B2} and M₆−I_{B2}) to deliver the output current of the input stage to the output nodes Vₒ₁ and Vₒ₂, respectively. This topology offers a wide signal swing at the output. The output common mode feedback block (CMFB) may be is inserted to create a CM feedback loop to stabilize the output CM level of Vₒ₁ and Vₒ₂. VB is to bias the common gate voltage of M₅ and M₆.

Apart from functioning as the differential transconductor 23, the source-coupled transistor pairs M₁-M₂ and M₃-M₄ are also able to detect CM voltage fluctuations, and shift them to appear at the common source terminals of the two transistor pairs as V_{cmH} and V_{cmL}, respectively. The CM amplifier 27, which may be formed by formed by C_{ci}, C_{cf}, R_{cf} and the transconductor A_{C}, will perform CM signal amplification with a gain of −K=−2C_{ci}/C_{cf}. As in the traditional way of designing an amplifier circuit for a neuromodulation device, the resistor R_{cf} can be made by a MOS pseudo-resistor. Thus, together with a small C_{cf}, a high-pass cut-off frequency below 1 Hz can be achieved within a tiny chip area. Besides, the transconductor A_{C} only contributes CM noise that can be cancelled out in the differential mode. It can thus be designed in a compact and low-power fashion. Therefore, a large K can be achieved within a reasonable area.

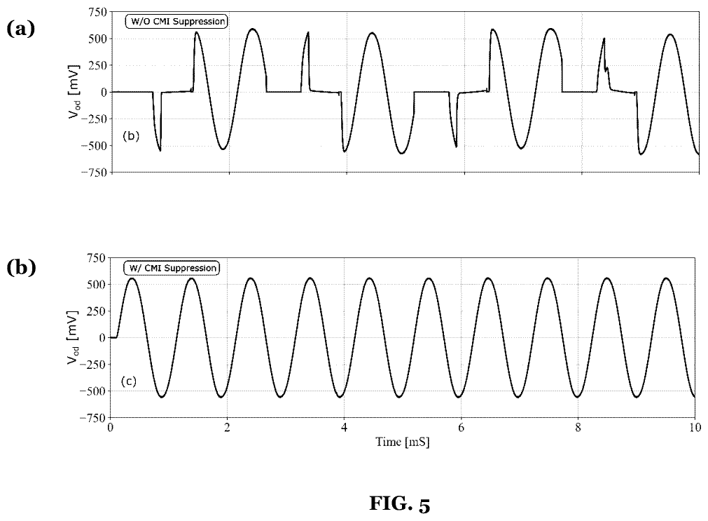

FIG. 5 compares exemplary output voltages of an amplifier circuit without CMI suppression (shown in FIG. 1(a)) and an amplifier circuit 20 comprising the CM amplifier 27 according to this disclosure. The comparison shows that a large CMI will exceed the CMR of the exemplary amplifier circuit, which may shutdown the amplifier circuit as visible in FIG. 5(a). The amplifier circuit 20 of this disclosure is more robust, as the CM is suppressed, and brings back a clean output signal in FIG. 5(b).

In particular, for the results of FIG. 5, a stimulation-like waveform of V_{CM} (CM voltage signal) was applied together with a sinusoidal V_{id} (DM voltage signal) to the amplifier circuits, and the relevant responses are shown in FIGS. 5(a) and (b), respectively. FIG. 5(a) shows the output voltage of the exemplary amplifier circuit without the CM amplifier 27 and without capacitive feedback via C_{CO} over time. At the time that the magnitude of Vᵢ₁ is approximately 200 mV, the amplifier circuit fails to provide any output signal. This implies, for instance, that reading a neural signal at a stimulation moment would not be possible. On the other hand, with the help of the CM amplifier 27 and the capacitive feedback via C_{CO}, the proper sinusoidal output voltage signal can be obtained, as shown in FIG. 5(b). Recording a neural signal during stimulation is possible in this case, i.e., with the amplifier circuit 20 of this disclosure.

Notably, for the amplifier circuit 20 used in FIG. 5(b), a 500 Hz sinusoidal V_{CM} with a 200 mVₚ amplitude was used. The main amplifier consumed 1.5 μA and its parameters were set to Cᵢₙ=2.1 pF, C_{f}=15 fF. The resistors R_{f} and R_{cf} were identically made by MOS pseudo-resistors. For the CM amplifier 27, C_{ci}=0.6 pF, C_{cf}=15 fF, C_{co}=0.6 pF, and Ac is formed by a simple wide-swing operational transconductance amplifier (OTA), which consumes 600 nA. V_{iCM} is could be suppressed to 2 mVₚ (i.e., 100 times smaller than V_{CM}).

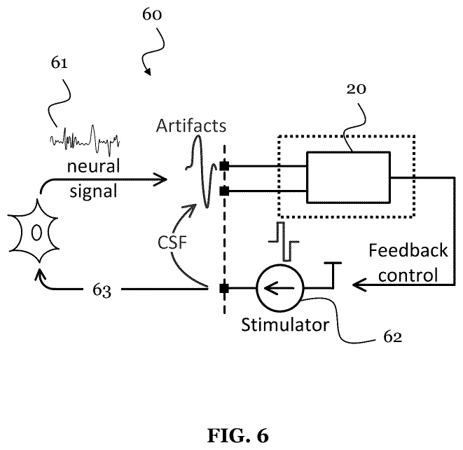

FIG. 6 shows an exemplary neuromodulation device 60 according to this disclosure. The neuromodulation device 60 is configured to measure one or more biological electrical signals 61 in response to a neural modulation of biological tissue. For measuring the one or more biological signals, the neuromodulation device 60 comprises at least one amplifier circuits 20 according to this disclosure, for instance, as described in the FIGS. 2-4.

The neuromodulation device 60 may further comprises at least one stimulator 62, which is configured to generate a stimulation signal 63, which can cause the neural modulation of the biological tissue. The stimulation signal 63 may be provided by a neural interface of the neuromodulation device 60 to the biological tissue. For instance, the neural interface may be a bi-directional neural interface, which is further configured to receive the one or more biological electrical signals 61 in response to the neural stimulation, and to provide them to the one or more amplifier circuits 20.

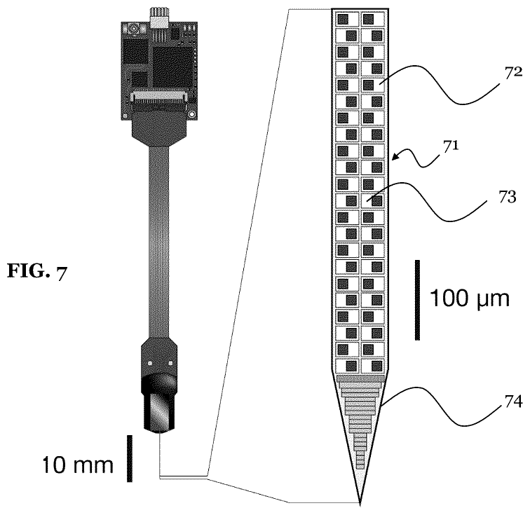

For example, as shown in FIG. 7, the neuromodulation device 60 may comprise a bi-directional neural interface comprising at least one needle 74. Moreover, the neuromodulation device 60 may comprise an array 71 of electrodes. The array 71 may include a first set of electrodes 72 and a second set of electrodes 73, which may be of a different kind or design. In particular, each electrode 72 of the first set may be connected to a respective amplifier circuit 20 of the one or more amplifier circuits 20 of the neuromodulation device 60. Each electrode 73 of the second set may be configured to cause a respective neural modulation of the biological tissue. For instance, each electrode 72 of the first set may comprise at least one amplifier circuit 20, and may be used for measuring (recording) an electrical biological signal 61. Each electrode 73 of the second set may comprises at least one stimulator 62, and may be used to generate a stimulation signal 63 to the interface, in this case comprising the needle 74.

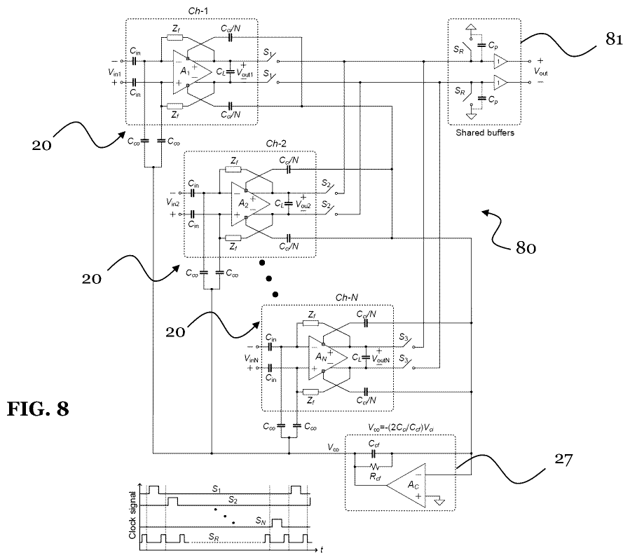

FIG. 8 shows a multichannel recording arrangement 80 according to this disclosure. The recording arrangement 80 comprises multiple of the amplifier circuits 20 according to this disclosure, for example, as shown in FIGS. 2-4. Each amplifier circuit 20 may be associated with one of multiple channels. The amplifier circuits 20 share a single CM amplifier 27. The CM amplifier 27 may be considered being a part of each amplifier circuit 20. The CM amplifier 27 is configured to provide, for each of the amplifier circuits 20, a respective inverted CM voltage signal 28 to the non-inverting input and the inverting input of the transconductor 23 of that amplifier circuit 20.

The shared CM amplifier 27 may improve the area per channel in the multichannel topology, since it can be shared among many recoding channels. This sharing is possible under the assumption that all recording electrodes (first set of electrodes 72) are placed in the nearby neighborhood and individually suffer from a comparable amount of CMI.

To achieve the same CM feedback gain (i.e., the same degree of CM suppression) for all N channels of the multichannel recording arrangement 80, only the one CM amplifier 27, including one transconductor Ac, but including N pairs of C_{ci}/N (the same area occupied by one pair of C_{ci}) and a pair of buffers can be used here. A pair of C_{co} is still used for each channel. This topology also offers weighted adjustment for each channel individually by adjusting the C_{co} value. This is useful, if the strength of the CMI appearing on each channel is known beforehand. The CM amplifier 27 may perform summing average of CMI and the required C_{EQ} for each channel, and can be adjusted via the value of C_{co} according to (1) and the known CMI.

The multichannel recording arrangement 80 of FIG. 8 may further comprises one or more shared buffers 81 (which may be used as ADC drivers), which are connected to the multiple amplifier circuits 20 and to the single CM amplifier 27. The output of each amplifier circuit 20 is connected by a respective switch S₁, S₂, S₃ . . . S_{N} to the shared buffers 81. The outputs of all channels (1 to N) may be multiplexed by switches S₁ to S_{N}, and may be controlled by the clock signals shown in timing diagram at the bottom of FIG. 8. That is, the recording arrangement 80 is configured to sequentially operate the switches S₁ . . . S_{N}, which causes the multiple amplifier circuits 20 to sequentially output their respective current signal, and causes the CM amplifier 27 to sequentially provide the respective inverted CM voltage signal 28 to the respective transconductor 23 of one of the amplifier circuits 20.

After the buffers 81 finish delivering a signal of one channel, further switches S_{R}(reset switches) may be turned on for a short time, in order to erase memories stored on parasitic capacitances Cₚ, before the buffers 81 receive the signal again from the next consecutive channel. This resetting mechanism helps minimizing channel crosstalk. By sharing the buffers 81, a total power and area consumed and occupied by the buffers 81 will be divided by N when per-channel power and area are calculated.

An example embodiment of this disclosure is further a multichannel neuromodulation device, which comprises the multichannel recording arrangement 80 shown in FIG. 8. In addition to the recording arrangement 80, this multichannel neuromodulation device may accordingly comprise at least one least one stimulator, which is configured to generate a stimulation signal. For instance, the multichannel neuromodulation device may be similar to the neuromodulation device 60 of FIG. 6 having multiple channels for recording. For example, the neuromodulation device 60 shown in FIG. 6 may comprise the recording arrangement 80 shown in FIG. 8.

The multichannel scheme of FIG. 8 was evaluated for a 16-channel (N=16) recording arrangement 80, to verify that the CM amplifier 27 can be shared. A stimulation-like waveform of V_{CM} together with sinusoidal V_{id} (as described above with respect to FIG. 5)) was applied to all channel inputs. Similar results as shown in FIG. 5 were obtained, which proves that the shared CM amplifier 27 is well functional.

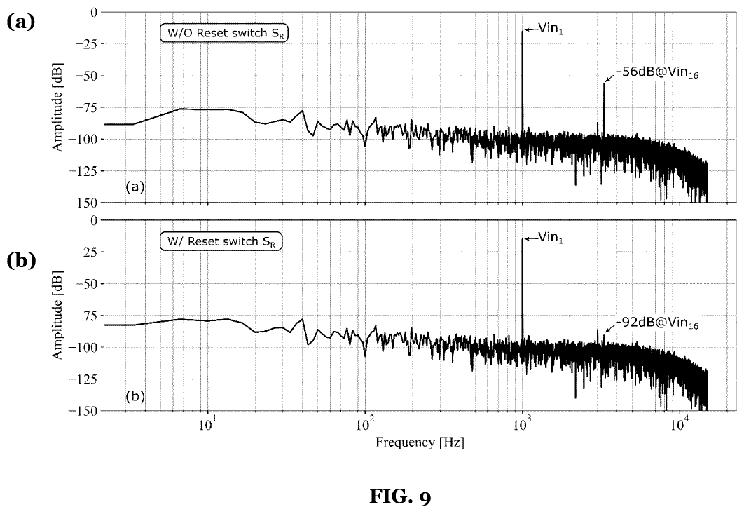

Sharing, for instance, two buffers 81 among the 16 outputs with negligible channel-to-channel crosstalk is also possible thanks to the reset switches S_{R}. FIG. 9 shows a Discrete Fourier Transform (DFT) of a first channel with and without reset mechanism. A 1 kHz 3 mVₚ input voltage signal was applied to the first channel. The rest of the channel inputs received the same signal amplitude, but different frequencies that are not set to be clearly visible in frequency domain (not located at any harmonics of the first channel's input signal). FIG. 9(a) shows, for the case that S_{R} are always turned off (Cₚ are not discharged), that the first channel's output comprises fundamental component at 1 kHz, its 3^{rd} harmonic component, and the output of the previous recorded channel, i.e., channel 16 (as a crosstalk component with −56 dB magnitude at 3.3 kHz). This crosstalk component was stored on Cₚ and added to the output signal of the first channel 1 after S₁ is closed. This crosstalk can be minimized by the controlling of the reset switches S_{R} according to the timing diagram in FIG. 8. The result is obtained in FIG. 9(b). The memory of channel 16 stored on Cₚ is erased almost completely. The crosstalk component is thus reduced from −56 dB to −92 dB.

In summary of this disclosure, an amplifier circuit 20, a neuromodulation device 60, and a recording arrangement 80 are provided. The amplifier circuit 20 is configured to suppress a CM voltage signal in the input voltage signal 22, and consequently to reduce CMI, while not degrading the input impedance.

What is claimed is:

1. An amplifier circuit for amplifying a biological electrical signal, the amplifier circuit comprising:
an input stage with two input terminals, configured to receive an input voltage signal, which is based on the biological electrical signal and comprises a common mode, CM, voltage signal and a differential mode, DM, voltage signal;
a differential transconductor comprising a non-inverting input and an inverting input, each connected to one of the two input terminals, and comprising an output for providing an output current signal based on the DM voltage signal;
wherein the transconductor is configured to provide a first CM voltage signal, which is tapped after the non-inverting input, and a second CM voltage signal, which is tapped after the inverting input; and
a CM amplifier configured to combine the first CM voltage signal with the second CM voltage signal to obtain a combined CM voltage signal, amplify the combined CM voltage signal with an inverting gain to obtain an inverted CM voltage signal, and provide the inverted CM voltage signal to the non-inverting input and the inverting input of the transconductor.

2. The amplifier circuit of claim 1, comprising:
a first capacitive feedback line configured to feedback the inverted CM voltage signal from the CM amplifier to the non-inverting input of the transconductor; and
a second capacitive feedback line configured to feedback the inverted CM voltage signal from the CM amplifier to the inverting input of the transconductor;
wherein each of the first and the second capacitive feedback line comprises a first capacitor.

3. The amplifier circuit of claim 2, wherein:
each of the non-inverting input and the inverting input of the transconductor is connected to one of the two input terminals of the input stage via a respective second capacitor; and
the second capacitor has a higher capacitance than the first capacitor.

4. The amplifier circuit of claim 1, wherein:
each of the non-inverting input and the inverting input of the transconductor is connected to one of the two input terminals of the input stage via a respective second capacitor; and
the second capacitor has a higher capacitance than the first capacitor.

5. The amplifier circuit of claim 1, wherein the transconductor comprises:
a first transistor connected with its gate to the non-inverting input and coupled with its source to a supply voltage;
a second transistor connected with its gate to the inverting input, connected with its source to the source of the first transistor, and coupled with its source to the supply voltage;
a third transistor connected with its gate to the non-inverting input, connected with its drain to the drain of the first transistor, and coupled with its source to a ground voltage; and
a fourth transistor connected with its gate to the inverting input, connected with its drain to the drain of the second transistor, connected with its source to the source of the third transistor, and coupled with its source to the ground voltage;
wherein the first CM voltage signal is tapped between the sources of the first and the second transistor, and the second CM voltage signal is tapped between the sources of the third and the fourth transistor.

6. The amplifier circuit of claim 5, configured such that a tail current of the source-connected third and fourth transistor is higher than a tail current of the source-connected first and second transistor in use of the amplifier circuit.

7. The amplifier circuit of claim 6, wherein the CM amplifier comprises:
a summing junction at which the first CM voltage signal and the second CM voltage signal are combined into the combined CM voltage signal;
wherein each of the first CM voltage signal and the second CM voltage signal is connected to the summing junction via a respective third capacitor.

8. The amplifier circuit of claim 7, wherein:
the CM amplifier further comprises a gain circuit including a second transconductor, a fourth capacitor and a first resistor; and
the gain circuit is configured to amplify the combined CM voltage signal with the inverting gain to produce the inverted CM voltage signal.

9. The amplifier circuit of claim 1, wherein the CM amplifier comprises:
a summing junction at which the first CM voltage signal and the second CM voltage signal are combined into the combined CM voltage signal;
wherein each of the first CM voltage signal and the second CM voltage signal is connected to the summing junction via a respective third capacitor.

10. The amplifier circuit claim 1, further comprising:
a DM feedback circuit configured to provide a feedback voltage signal, which is based on the output current signal of the transconductor, to the non-inverting input and the inverting input of the transconductor.

11. The amplifier circuit of claim 10, wherein:
the DM feedback circuit comprises a first capacitive feedback path and a second capacitive feedback path; and
each capacitive feedback path comprises a respective fifth capacitor that is connected in parallel to a respective second resistor.

12. A neuromodulation device for measuring one or more biological electrical signals in response to a neural modulation of biological tissue, wherein the neuromodulation device comprises one or more amplifier circuits including:
an input stage with two input terminals, configured to receive an input voltage signal, which is based on the biological electrical signal and comprises a common mode, CM, voltage signal and a differential mode, DM, voltage signal;
a differential transconductor comprising a non-inverting input and an inverting input, each connected to one of the two input terminals, and comprising an output for providing an output current signal based on the DM voltage signal;
wherein the transconductor is configured to provide a first CM voltage signal, which is tapped after the non-inverting input, and a second CM voltage signal, which is tapped after the inverting input; and
a CM amplifier configured to combine the first CM voltage signal with the second CM voltage signal to obtain a combined CM voltage signal, amplify the combined CM voltage signal with an inverting gain to obtain an inverted CM voltage signal, and provide the inverted CM voltage signal to the non-inverting input and the inverting input of the transconductor.

13. The neuromodulation device of claim 12, further comprising:
an array of electrodes comprising a first set of electrodes and a second set of electrodes, wherein each electrode of the first set is connected to a respective amplifier circuit of the one or more amplifier circuits, and wherein each electrode of the second set is configured to cause a respective neural modulation of the biological tissue.

14. The neuromodulation device of claim 13, wherein each electrode of the second set of electrodes comprises a stimulator, which is configured to provide a stimulation signal to cause the neural modulation of the biological tissue.

15. The neuromodulation device of claim 14, comprising:
multiple of the amplifier circuits;
wherein the amplifier circuits share a single CM amplifier; and
the CM amplifier is configured to provide, for each of the amplifier circuits, a respective inverted CM voltage signal to the non-inverting input and the inverting input of the transconductor of that amplifier circuit.

16. The neuromodulation device of claim 12, comprising:
multiple of the amplifier circuits;
wherein the amplifier circuits share a single CM amplifier; and
the CM amplifier is configured to provide, for each of the amplifier circuits, a respective inverted CM voltage signal to the non-inverting input and the inverting input of the transconductor of that amplifier circuit.

17. The neuromodulation device of claim 16, further comprising:
one or more shared buffers connected to the multiple amplifier circuits and to the single CM amplifier, wherein the output of each amplifier circuit is connected by a respective switch to the shared buffers;
wherein the neuromodulation device is configured to sequentially operate the switches, which causes the multiple amplifier circuits to sequentially output their respective current signal and causes the CM amplifier to sequentially provide the respective inverted CM voltage signal to the respective transconductor of one of the amplifier circuits.

18. The neuromodulation device of claim 17, further comprising:
a bi-directional neural interface comprising at least one needle;
wherein the neural interface is configured to provide the neural stimulation to the biological tissue, to receive the one or more biological electrical signals, and to provide them to the one or more amplifier circuits.

19. The neuromodulation device of claim 12, further comprising:
a bi-directional neural interface comprising at least one needle;
wherein the neural interface is configured to provide the neural stimulation to the biological tissue, to receive the one or more biological electrical signals, and to provide them to the one or more amplifier circuits.
